(19) United States
(12) Patent Application Publication
(10) Pub. No.: US 2008/0188786 A1
(43) Pub. Date: Aug. 7, 2008

(54) Orthopaedic compress support

(75) Inventor: Shawn J. Hickling, (US)

Correspondence Address: SHAWN J. HICKLING, 9580 Mamota Way, Cypress, CA, 90630, US

(73) Assignee: ActiveWrap Inc

(21) Appl. No.: 11/590,896

(22) Filed: Nov. 1, 2006

(51) Int. Cl. A61F 5/00

(52) U.S. Cl. 602/26; 602/2

(57) ABSTRACT

An orthopaedic compress support is used on the leg, including the knee and the various muscle groups of the leg. The support has a panel of flexile material including a concave main body and two wings projecting from one side of the main body. The main body is shaped to cover that portion of the limb to be treated while the wings wrap around and encircle the limb. The inner surface of the panel is a pile fabric. Two elastically stretchable straps are fastened to respective ones of the wings. Each strap has an outer surface of pile fabric and a tab of hook material at the free end to engage the pile fabric when the strap is wrapped around the leg under tension to provide compression and also joint support where used on the knee. Two flat pouches of a textile fabric have openings for the insertion and removal of a thermal treatment medium, e.g. gel packs or ice. The pouches are fastened to the inside pile fabric of the panel by patches of hook material secured to one surface of each pouch so that each pouch may be secured at any desired position and orientation to the inner surface of the panel.

### FIELD OF THE INVENTION

The present invention relates to an orthopaedic compress support suitable for providing compressive support for the lower limb, including the knee, preferably in combination with thermal treatment.

### BACKGROUND

U.S. Pat. No. 5,743,867, granted Apr. 28, 1998, and commonly owned with the present application discloses a therapeutic wrapping for a joint, particularly the ankle. This wrapping includes a system for retaining thermal treatment pads on the interior of the wrap to provide thermal treatment and joint support. To accommodate its intended purpose as an ankle wrapping, the structure includes an opening to accommodate the protruding calcaneus or heel bone. Other wraps intended for joints of the upper and lower limbs are likewise especially configured to fit the joint in question with a central opening to accommodate the projecting bones of the joints. With the knee, the patella is usually left open.

### SUMMARY

The present invention is concerned with the provision of a structure with a more general applicability and that is suitable for treating most parts of the lower limb, including the knee, and the main musculature of the leg, for example the quadriceps and hamstring muscles.

According to one aspect of the present invention there is provided an orthopaedic compress support for use on a lower limb, the support comprising:

a panel of flexile material, the panel being shaped and arranged to cover a portion of the limb to be treated, the panel having an inner surface comprising a first pile fabric;

two elastically stretchable straps each having distal and proximal ends, the proximal ends being secured to positions spaced apart on the panel, each strap having an outer strap surface consisting of a second pile fabric;

a strap fastener having a first component which comprises the second pile fabric and a second component comprising a tab of hook material secured to an inner surface of each strap adjacent the distal end, the hook material being engageable with the pile fabric so as to hold the two components together;

a plurality of substantially flat pouches of flexile material, each pouch having opposite inner and outer surfaces and an opening for the insertion and removal of a thermal treatment medium; and

a pouch fastener system having a first component comprising the first pile fabric and a second component comprising a hook material secured to the outer surface of each pouch, the hook material being engageable with the first pile fabric to secure a pouch at any selected position and orientation on the inner surface of the panel.

A support of this configuration may be wrapped about any part of the leg, including the knee, and the straps wrapped about the leg under tension to apply compression to the desired portion of the leg. The straps can be fastened with various levels of tension to provide not only compression but joint support where used on the knee. Where thermal treatment (hot or cold) is desired, a thermal treatment medium may be inserted in one or more of the pouches, and those pouches may be attached to the inside of the support at whatever position and orientation is suitable.

The thermal treatment medium will normally be a “gel pack”, conventionally a plastic material envelope containing a high heat capacity gel. Where a cold gel pack is not readily available when wanted, the pouches can be filled with ice, either crushed or cubes.

Preferably, the panel includes a main body and two wings with the wings projecting from one side of the main body to wrap around the limb so that the panel substantially encircles the limb. The straps are secured at their proximal ends to respective ones of the wings, and may be wrapped circumferentially about the limb to apply compression thereto. Where desired, the straps may cross over, with each being fastened to the other. This may be particularly useful in applying compression to the knee in the area of the patella and supporting the joint.

Desirably, the main body of the panel is concave. This accommodates the pouches while allowing the support to conform to the shapes of various leg parts to be treated, including the knee, which is allowed to flex, a characteristic that is further enhanced by the wing configuration where the separation of the wings allows contraction of the wrapped support on one side.

It is preferred practice to form the pouches of a washable textile fabric. This not only allows the entire support to be washed when necessary, but also provides that all of the surfaces contacting the leg are a comfortable cloth material.

It has been determined that the support best achieves its objectives with two rectangular pouches of equal size dimensioned collectively to cover the entire inner surface of the main body of the panel. Each pouch has a closure for selectively closing its opening to retain the thermal medium.

### BRIEF DESCRIPTION OF THE DRAWINGS

In the accompanying drawings, which illustrate an exemplary embodiment of the present invention:

FIG. 1 is a plan view showing the inner side of an orthopaedic compress support;

FIG. 2 is a plan view showing the outer side of the support;

FIG. 3 is a section along line 3-3 of FIG. 1;

FIG. 4 is an illustration of a pouch, containing a gel pack as a thermal treatment medium, the pouch being partially broken away to show the gel pack and the pouch closure applied to a knee; and

FIG. 5 is an illustration of the support applied to a knee.

### DETAILED DESCRIPTION

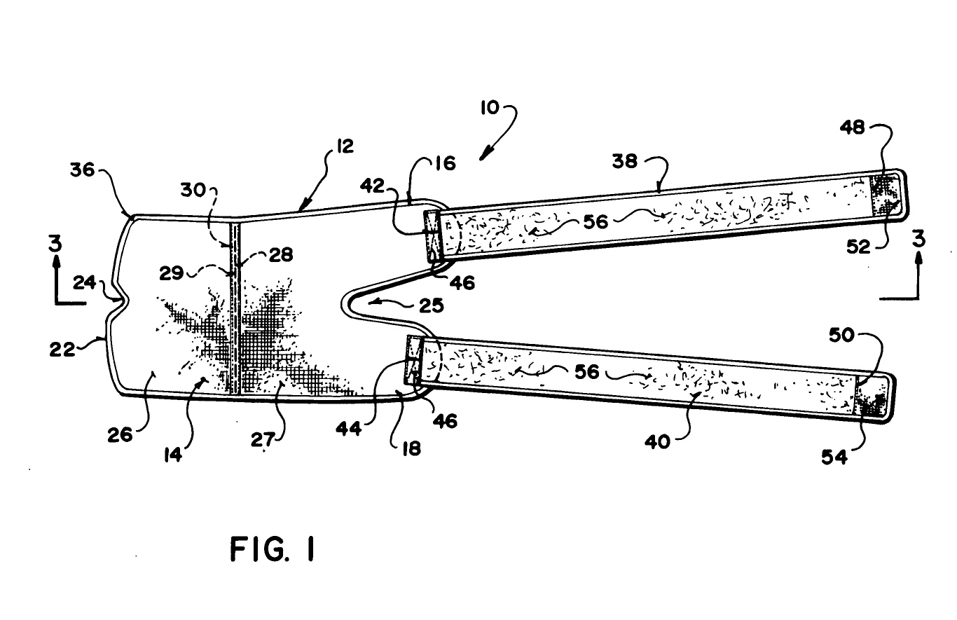

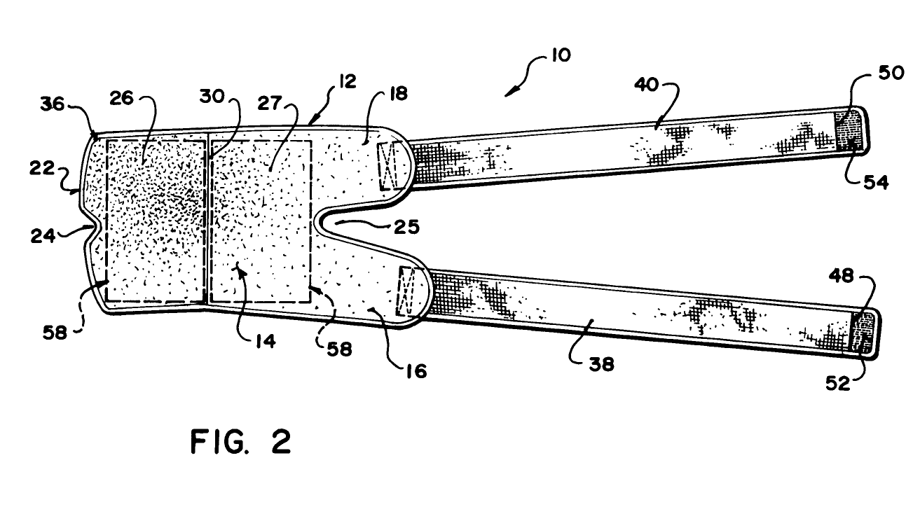

Referring to the accompanying drawings, there is illustrated an orthopaedic compress support 10. As illustrated most particularly in FIGS. 1 and 2, the support includes a panel 12 with a main body 14 of generally rectangular outline shape and two wings 16 and 18 extending outwardly from a common side of the main body 14. Each wing extends outwardly roughly one half the width of the main body. On the side 22 of the main body that is opposite the wings, there is a wide, short notch 24, rounded at each corner to limit stress concentrations and allowing the side 22 of the main body 14 to contract to conform to the shape of the parts to which the support may be applied. The space 25 between the wings 16 and 18 provides a similar function on the opposite side of the main body 14.

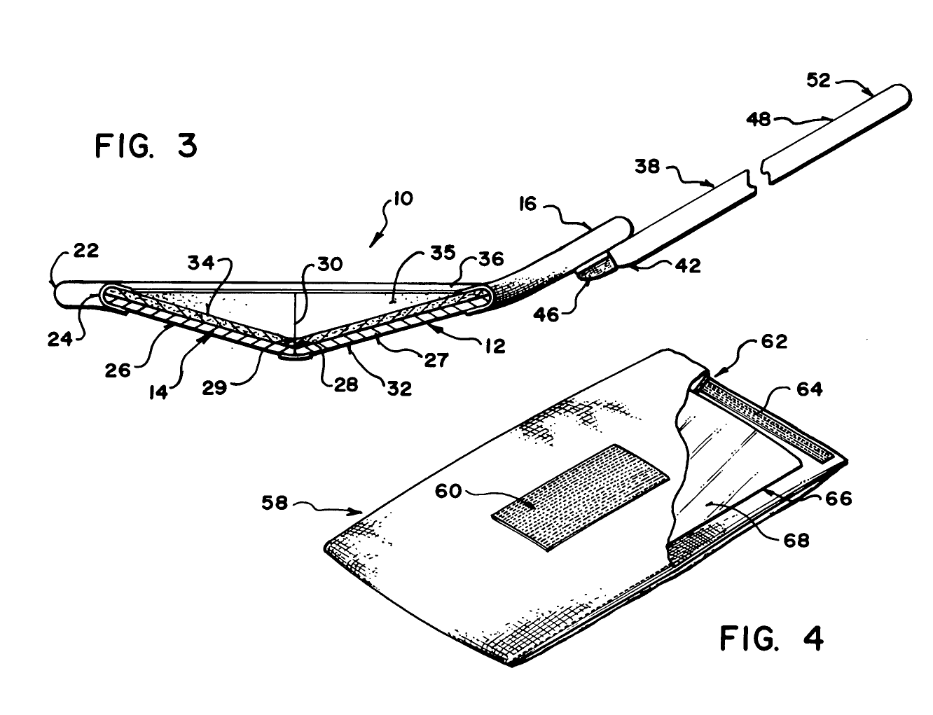

The main body 14 of the panel 12 is formed from two parts 26 and 27, with respective convex edges 28 and 29 stitched together along a seam 30 located centrally between the sides of the main body. This gives the main body 14 a “dished” or concave configuration as illustrated most particularly in FIG. 3.

The panel 12 is formed with an outer layer 32 of an elastic sheet material, for example neoprene, and an inner layer 34 of a textile fabric with an inner pile surface 35. The layers 32 and 34 are secured together along the periphery of the panel 12 by a stitched-on elastic edge binding 36.

The support includes two elastic compression straps 38 and 40 with their proximal ends 42 and 44 respectively secured to the outer sides of the wings 16 and 18 respectively. These proximal ends are secured by stitching through webbing 15 patches 46 overlying the strap ends and the wings. The straps 38 and 40 have distal ends 48 and 50 respectively, which have secured thereto tabs 52 and 54 respectively of a hook fastener material. The outer surface of each of the straps 38 and 40 is a pile fabric 56 engageable with the hook material tabs 52 and 54 to hold the strap ends in place. The length of each strap is roughly the same as the total width of the panel 12, including the wings.

The support also includes two textile fabric pouches 58. These are generally rectangular in shape, and each carries on one side a strip 60 of a hook material engageable with the pile surface of the inner layer 34 of the panel 12 to secure the pouch in place. The two pouches are dimensioned to together cover the inner surface of the main body 14 of the panel 12 as generally illustrated in broken line in FIG. 2. Each pouch has an opening 62 at one end that is closed by a hook and loop fastener 64.

Each pouch may contain a gel pack 66 as a thermal treatment medium. The gel packs conventionally consist of a sealed synthetic plastic envelope 68 containing a quantity of gel material 68 which has a high heat capacity and may be either heated or chilled to provide the desired treatment. With the pouch design, however, other thermal treatment media may be used, for example crushed ice, ice cubes or the grains that are sometimes packaged in fabric bags for use in heat treatment.

In use, the support is prepared by placing the desired thermal treatment medium in one or both of the pouches and fastening the pouch or pouches in the desired place on the inside of the panel 12 using the panel fastener consisting of the hook material strip 60 on the pouch and the pile inner surface 34 of the panel. The support is then wrapped around the limb with the pouches located to heat or cool the area to be treated. The straps are wrapped around the leg to apply compression and are fastened in place using the strap fasteners consisting of the pile outer surfaces of the straps and the co-operating hook material tabs at the distal ends of the straps.

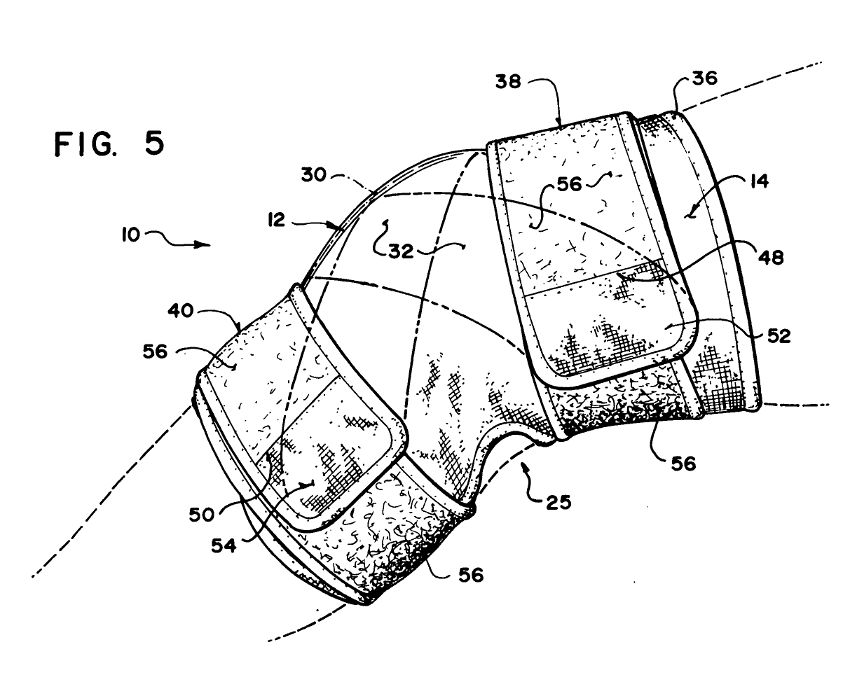

The straps may be wrapped circumferentially around the leg, with each strap being fastened to itself as shown in full lines in FIG. 5. Alternatively, the straps may cross over with each fastening to the other as shown in ghost line in FIG. 5.

The support may, if desired, be used for compression and support only, without a thermal treatment medium.

The concave configuration of the main body of the panel accommodates the pouches and their contents so as to conform to any portion of the lower limb, including the quadriceps, hamstring and knee. When applied to the knee, the wings 16 and 18 wrap around the posterior of the leg above and below the knee, allowing the knee to flex.

As will be apparent to those skilled in the art various modifications of my invention may be made without departing from the spirit and scope of the invention. It is therefore to be understood that the invention is not limited to the specific embodiment described in the foregoing. The invention is to be considered limited solely by the scope of the appended claims.

Embodiments of the invention in which an exclusive property or privilege is claimed are defined as follows:

1. An orthopaedic compress support for use on a lower limb, the support comprising:
a panel of flexile material, the panel being shaped and arranged to cover a portion of the limb to be treated, the panel having an inner surface comprising a first pile fabric;
two elastically stretchable straps each having distal and proximal ends, the proximal ends being secured to positions spaced apart on the panel, each strap having an outer strap surface consisting of a second pile fabric;
a strap fastener having a first component which comprises the second pile fabric and a second component comprising a tab of hook material secured to an inner surface of each strap adjacent the distal end, the hook material being engageable with the pile fabric so as to hold the two components together;
a plurality of substantially flat pouches of flexile material, each pouch having opposite inner and outer surfaces and an opening for the insertion and removal of a thermal treatment medium; and
a pouch fastener system having a first component comprising the first pile fabric and a second component comprising a hook material secured to the outer surface of each pouch, the hook material being engageable with the first pile fabric to secure a pouch at any selected position and orientation on the inner surface of the panel.

2. A support according to claim 1 wherein the panel includes a main body and two wings, the wings projecting from the main body to wrap around and encircle the limb, the straps being secured at their proximal ends to respective ones of the wings.

3. A support according to claim 2 wherein the wings project from a common side of the central portion.

4. A support according to claim 1 wherein the panel is concave.

5. A support according to claim 1 wherein the main body of the panel is concave.

6. A support according to claim 1 wherein each of the pouches comprises a textile fabric pouch.

7. A support according to claim 1 wherein the plurality of pouches consists of two pouches dimensioned to collectively cover the inner surface of the main body of the panel.

8. A support according to claim 7 wherein the pouches are equal in size.

9. A support according to claim 1 wherein the pouches are rectangular.

10. A support according to claim 1 wherein each pouch comprises a closure for selectively closing the opening thereof.

11. A support according to claim 1 wherein the thermal treatment medium comprises a gel pack.
